(19) United States
(12) Patent Application Publication
(10) Pub. No.: US 2004/0146473 A1
(43) Pub. Date: Jul. 29, 2004

(54) Dispersions of polymers in silicone medium, and compositions comprising them

(75) Inventor: Bertrand Lion, Luzarches (FR)

Correspondence Address: LERNER, DAVID, LITTENBERG,, KRUMHOLZ & MENTLIK, 600 SOUTH AVENUE WEST, WESTFIELD, NJ, 07090, US

(73) Assignee: L'OREAL, Paris (FR)

(21) Appl. No.: 10/735,320

(22) Filed: Dec. 12, 2003

Related U.S. Application Data
(60) Provisional application No. 60/445,187, filed on Feb. 5, 2003.

(30) Foreign Application Priority Data
Dec. 12, 2002 (FR) 0215739

(51) Int. Cl. A61K 7/06; A61K 7/11

(52) U.S. Cl. 424/70.12; 424/70.16

(57) ABSTRACT

The invention relates to self-stabilized dispersions of polymer particles in a silicone medium, and to cosmetic or pharmaceutical compositions comprising the said polymer particle dispersions.

### CROSS-REFERENCE TO RELATED APPLICATIONS

The present application claims the benefit of French Patent Application No. FR 02 15739, the disclosure of which is incorporated by reference herein.

### BACKGROUND OF THE INVENTION

The present invention relates to stable dispersions of particles formed from acrylic polymers in a silicone medium, and also to the use of these dispersions in cosmetic compositions and to the compositions thus obtained.

It is known practice in cosmetics to use dispersions of polymer particles in organic media, as film-forming agents in various cosmetic formulations, such as mascaras, eyeliners, eye shadows or nail varnishes.

Thus, European patent application EP-A-0 749 747 describes a composition comprising a dispersion of polymer particles that are insoluble in a non-aqueous medium, the said dispersion being stabilized by adding stabilizing polymers. The stabilizing polymers according to that document bond non-covalently via physical interactions with the insoluble polymers mentioned above.

However, this type of composition has the following drawbacks: it requires the addition to the non-aqueous medium of an amount of “stabilizing” polymers that is higher than that effectively bonded to the insoluble polymer particles, in order to obtain a relatively stable dispersion of the said particles. However, during the addition of adjuvants to these compositions, such as pigments, there is a tendency for some of the stabilizing polymers to become desorbed from the insoluble polymer particles and to combine with the said adjuvants, which contributes towards destabilizing the dispersion, especially by forming aggregates between the polymer particles.

Document JP 11 181 003 describes polymers that are suitable for forming solid particles without addition of stabilizing polymers; however, these particles are unstable in non-aqueous organic media.

### SUMMARY OF THE INVENTION

The Applicant has discovered, surprisingly, novel polymers that are capable of forming stable particles in a silicone medium, without adding stabilizing polymers.

Thus, one aim of the present invention is to provide a dispersion, in a silicone organic medium, of self-stabilized individual polymer particles, preferably solid polymer particles, the said dispersion being free of particle aggregates and of insoluble sediments, visually, for example, after leaving the dispersion to stand for one day (24 hours) at room temperature (approximately 25° C.).

Moreover, it has been found that certain dispersions of polymer particles in a non-aqueous medium, prepared in the prior art, show poor resistance to sebum, or to fats (for example to oil). This may be prohibitive for certain categories of cosmetic compositions, in particular for makeup compositions such as foundations or lipsticks, which show poor resistance over time.

An aim of the present invention is thus also to propose a composition with improved resistance, especially to fats and to sebum, and also good transfer-resistance properties, while at the same time having no problem of tack.

A first subject of the present invention is thus a dispersion, in a non-aqueous, silicone medium, of solid particles comprising at least one acrylic polymer comprising a skeleton that is insoluble in the said medium, and a portion that is soluble in the said medium, comprising side chains covalently bonded to the said skeleton, wherein the said polymer is obtained by polymerization of a polymerizable mixture comprising:

a first C₁-C₃ alkyl (meth)acrylate monomer, alone or as a mixture of C₁-C₃ alkyl (meth)acrylate monomers, optionally in the presence of one or more additional monomers chosen from acrylic acid, methacrylic acid and alkyl (meth)acrylates of formula (I), and the salts thereof, to form the said insoluble skeleton; and

at least one silicone macromonomer comprising an end group that reacts during the polymerization to form the said side chains, the said macromonomer having a weight-average molecular mass of at least 200 and representing 0.05% to 20% by weight of the polymer.

Another subject of the invention is a cosmetic or pharmaceutical composition comprising, in a cosmetically or pharmaceutically acceptable medium, a dispersion as defined above.

The dispersions according to the invention are thus free of stabilizing polymer, such as those described in EP 749 747, and the polymers according to the invention are therefore not surface-stabilized with such additional stabilizing polymers. The dispersions according to the invention thus comprise a non-aqueous, silicone medium.

### DETAILED DESCRIPTION OF THE INVENTION

The different subject matter of the invention will now be detailed. All of the meanings and definitions of the compounds given below are valid for all of the subject matter of the invention.

The expression “non-aqueous medium” means a medium comprising one or more silicone compounds as defined below, the said medium possibly containing up to 1% by weight of water.

The expression “silicone medium” means a medium comprising one or more silicone compounds as defined below, that is to say a medium in which the said silicone compounds represent at least 50% by weight, especially from 50% to 100% by weight, for example from 60% to 99% or even from 65% to 95% by weight, of all the “silicone compounds+possible non-silicone compounds+possible water” liquid constituents making up the said medium.

The said medium may thus optionally comprise non-silicone compounds that may be present in a maximum amount of 50% by weight, especially from 0 to 40% by weight or even from 1% to 35% by weight, and even further 5-30% by weight relative to the total weight of the medium. Preferably, the silicone medium is liquid.

Among the silicone compounds that may be present in the said silicone medium, mention may be made of non-aqueous silicone liquid compounds having a global solubility parameter according to the Hansen solubility space of less than or equal to 17 (MPa)^{1/2}.

The global solubility parameter δ according to the Hansen solubility space is defined in the article “Solubility parameter values” by Eric A. Grulke in the book “Polymer Handbook” 3rd Edition, Chapter VII, p. 519-559, by the relationship:

δ=(d_{D}²+d_{P}²+d_{H}²)^{1/2}

in which:

d_{D} characterizes the London dispersion forces arising from the formation of dipoles induced during molecular impacts;

d_{P} characterizes the Debye interaction forces between permanent dipoles; and

d_{H} characterizes the forces of specific interactions (such as hydrogen bonding, acid/base, donor/acceptor, etc.).

The definition of solvents in the solubility space according to Hansen is described in the article by C. M. Hansen: “The three-dimensional solubility parameters”, J. Paint Technol. 39, 105 (1967).

In particular, mention may be made of optionally branched, volatile and/or non-volatile silicone oils.

The term “volatile oil” means an oil that is capable of evaporating from the skin or the lips in less than one hour, especially having a vapour pressure, at room temperature and atmospheric pressure, ranging from 10⁻³ to 300 mmHg (0.13 Pa to 40,000 Pa).

As volatile silicone oils that may be used in the invention, mention may be made of linear or cyclic silicones containing from 2 to 7 silicon atoms, these silicones optionally comprising alkyl or alkoxy groups containing from 1 to 10 carbon atoms. In particular, mention may be made of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane and decamethyltetrasiloxane, and mixtures thereof.

Non-volatile silicone oils that may be mentioned include non-volatile polydialkylsiloxanes, such as non-volatile polydimethylsiloxanes (PDMSs); polydimethylsiloxanes comprising alkyl, alkoxy or phenyl groups containing from 2 to 24 carbon atoms, which are pendent or at the end of a silicone chain; phenylsilicones, for instance phenyl trimethicones, phenyl dimethicones, phenyltrimethylsiloxydiphenylsiloxanes, diphenyl dimethicones, diphenylmethyldiphenyltrisiloxanes and polymethylphenylsiloxanes; polysiloxanes modified with fatty acids (especially of C₈-C₂₀), fatty alcohols (especially of C₈-C₂₀) or polyoxyalkylenes (especially polyoxyethylene and/or polyoxypropylene); amino polysiloxanes; polysiloxanes containing hydroxyl groups; and fluoro polysiloxanes containing from 1 to 12 carbon atoms comprising a fluoro group that is pendent or at the end of a silicone chain, all or some of the hydrogens of which are replaced with fluorine atoms; and mixtures thereof.

Among the non-silicone compounds that may be present, in small amount, in the silicone medium, mention may be made of non-aqueous non-silicone liquid compounds with a global solubility parameter according to the Hansen solubility space of less than 17 (MPa)^{1/2}; monoalcohols with a global solubility parameter according to Hansen solubility space of less than or equal to 20 (MPa)^{1/2}; and mixtures thereof.

Among the non-aqueous non-silicone liquid compounds, mention may be made of liquid fatty substances, especially oils, which may be chosen from natural or synthetic, carbon-based, hydrocarbon-based and fluoro oils, which are optionally branched, alone or as a mixture. Among these oils, mention may be made of plant oils formed from fatty acid esters and from polyols, in particular triglycerides, such as sunflower oil, sesame oil or rapeseed oil, or esters derived from acids or alcohols containing a long chain (i.e. a chain containing from 6 to 20 carbon atoms), in particular the esters of formula RCOOR in which R represents a higher fatty acid residue containing from 7 to 19 carbon atoms and R′ represents a hydrocarbon-based chain containing from 3 to 20 carbon atoms, such as palmitates, adipates and benzoates, in particular diisopropyl adipate. Mention may also be made of linear, branched and/or cyclic alkanes which may be volatile, and in particular liquid paraffin, liquid petroleum jelly or hydrogenated polyisobutylene, isododecane or “Isopars”, volatile isoparaffins. Mention may also be made of linear, branched or cyclic esters containing more than 6 carbon atoms, ethers containing more than 6 carbon atoms, and ketones containing more than 6 carbon atoms.

The expression “monoalcohols having a global solubility parameter according to the Hansen solubility space of less than or equal to 20 (MPa)^{1/2}” means aliphatic fatty monoalcohols containing 6 to 30 carbon atoms, the hydrocarbon-based chain not comprising a substitution group, among which are oleyl alcohol, decanol, dodecanol, octadecanol and linoleyl alcohol.

The choice of monomers forming the skeleton of the polymers, the choice of macromonomers, the molecular weight of the polymer and of the side chains and the proportion of the monomers and of the macromonomers in the polymer, will be made as a function of the silicone medium so as to obtain a polymer particle dispersion that is stable in the said medium, this choice being made by a person skilled in the art.

According to the invention, the term “stable dispersion” means a dispersion that is not liable to form a solid deposit or to undergo liquid/solid phase separation especially after centrifugation, for example at 4000 rpm for 15 minutes.

The acrylic polymers forming the particles in dispersion thus comprise a skeleton that is insoluble in the said medium and a portion that is soluble in the said medium.

These polymers may be in various forms, in particular in the form of random polymers.

According to the invention, the term “acrylic polymer” means a polymer that is obtained by polymerization of a polymerizable mixture comprising:

a first monomer of C₁-C₃ alkyl (meth)acrylate type, alone or as a mixture;

optional additional monomers chosen from acrylic acid, methacrylic acid, alkyl (meth)acrylates of formula (I), and mixtures thereof; and

one or more macromonomers, in a given non-aqueous silicone medium or in a polymerisation medium.

Preferably, the first monomer, or mixture of first monomers, represents 50-100% by weight, especially 55 to 95% by weight or even 60 to 80% by weight of the mixture “first monomer(s) and optional additional monomer(s)”.

According to the invention, the term “macromonomer” means any polymer, specially oligomer, comprising, at only one of its ends, an end group, preferably polymerizable, capable of reacting with the monomers, during the polymerization reaction, to form side chains, the said group possibly being an ethylenically unsaturated group capable of undergoing free-radical polymerization with the monomers constituting the skeleton.

Preferably, the macromonomer is chosen from macromonomers whose homopolymer is soluble in the silicone medium under consideration, i.e. fully dissolved at a concentration of greater than or equal to 5% by weight and at room temperature in the said medium.

Thus, the polymers according to the invention are in the form of polymers that are insoluble in the medium under consideration, and comprise a skeleton (or main chain) consisting of a sequence of units, especially acrylic units, resulting from the polymerization especially of one or more acrylic monomers and of side chains (or grafts) derived from the reaction of the silicone macromonomers, the said side chains being covalently bonded to the said main chain.

The skeleton (or main chain) is insoluble in the medium under consideration, whereas the side chains (or grafts) are soluble in the said medium.

As a first monomer that may be used to constitute the insoluble skeleton of the polymer after polymerization, mention may be made, alone or as a mixture, of methyl acrylate, methyl methacrylate, ethyl acrylate, ethyl methacrylate, n-propyl acrylate, n-propyl methacrylate, isopropyl acrylate and isopropyl methacrylate.

Methyl acrylate, methyl methacrylate and ethyl methacrylate are most particularly preferred.

The additional monomers, optionally used with the said first monomer or mixture of first monomers, to make the insoluble skeleton of the acrylic polymer, after polymerization, are chosen, alone or as a mixture, from:

(meth)acrylic acid and its salts; and

the (meth)acrylates of formula (I) and salts thereof:

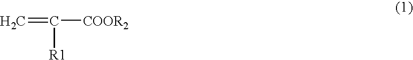

wherein:

R₁ is a hydrogen atom or a methyl group; and

R₂ is:

a linear or branched alkyl group containing from 1 to 6 carbon atoms, the said group containing one or more substituents chosen from one or two oxygen atoms, —OH, halogen atoms (F, Cl, Br or I), and —NR′R″, wherein R′ and R″, which may be identical or different, are linear or branched C₁-C₃ alkyls; or

a cyclic alkyl group containing from 3 to 6 carbon atoms, the said group optionally containing in its chain one or more oxygen atoms and optionally containing one or more substituents chosen from OH and halogen atoms (F, Cl, Br or I).

Examples of R₂ that may be mentioned include the methoxyethyl, ethoxyethyl, trifluoroethyl; 2-hydroxyethyl, 2-hydroxypropyl, dimethylaminoethyl, diethylaminoethyl or dimethylaminopropyl group.

Among these additional monomers that may be mentioned most particularly are, alone or as a mixture, methoxyethyl or ethoxyethyl (meth)acrylate; trifluoroethyl methacrylate; dimethylaminoethyl methacrylate; diethylaminoethyl methacrylate; 2-hydroxypropyl methacrylate; 2-hydroxyethyl methacrylate; 2-hydroxypropyl acrylate; 2-hydroxyethyl acrylate; and the salts thereof.

Acrylic acid and methacrylic acid are most particularly preferred.

Among the salts that may be mentioned are those obtained by neutralization of acid groups with mineral bases such as sodium hydroxide, potassium hydroxide or ammonium hydroxide, or organic bases such as alkanolamines, for instance monoethanolamine, diethanolamine, triethanolamine or 2-methyl-2-amino-1-propanol.

Mention may also be made of those formed by neutralization of ternary amine groups, for example using a mineral or organic acid. Among the mineral acids that may be mentioned are sulphuric, hydrochloric acid, hydrobromic acid, hydriodic acid, phosphoric acid and boric acid. Among the organic acids that may be mentioned are acids comprising one or more carboxylic, sulphonic or phosphonic groups. They may be linear, branched or cyclic aliphatic acids, or alternatively aromatic acids. These acids may also comprise one or more hetero atoms chosen from O and N, for example in the form of hydroxyl groups. Examples of organic acids include acetic acid, propionic acid, terephthalic acid, citric acid and tartaric acid.

It is understood that these non-polymerized first and additional monomers may be soluble in the medium under consideration, but become insoluble after polymerization in a suitable amount, which is the objective of the present invention.

The macromonomers constituting, after reaction, the side chains of the polymer according to the invention comprise, at the end of the chain, an end group capable of reacting during the polymerization with acrylic and vinyl monomers to form the said chains, the said end group being in particular a vinyl or (meth)acryloyloxy group (acrylate or methacrylate).

The macromonomers are preferably chosen from silicone macromonomers, in particular those whose homopolymers have a glass transition temperature (Tg) of less than or equal to 25° C., especially ranging from −100° C. to 25° C. and preferably ranging from −80° C. to 0° C. inclusive.

Preferably, the macromonomers according to the invention have a weight-average molecular mass (Mw) ranging from 200 to 100,000, preferably from 300 to 50,000, especially from 500 to 20,000, more preferably from 800 to 10,000, for example from 1,000 to 6,000.

Mention may be made in particular of polydimethylsiloxanes containing a monoacryloyloxy or monomethacryloyloxy end group, and especially those of formula (II):

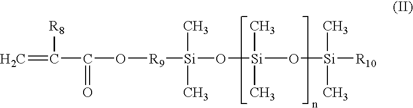

wherein:

R₈ denotes a hydrogen atom or a methyl group, preferably a methyl group;

R₉ represents a divalent linear or branched hydrocarbon group containing from 1 to 10 carbon atoms, the said group optionally containing one or two oxygen atoms; preferably R₉ represents ethylen, propylen or butylen;

R₁₀ represents a linear or branched alkyl group containing from 1 to 10 carbon atoms, especially from 2 to 8 carbon atoms; preferably R₁₀ represents methyl, ethyl, propyl, butyl or pentyl; and

n is an integer from 1 to 300, especially from 3 to 200, preferably from 5 to 100.

It is possible to mention monomethacryloyloxypropyl polydimethylsiloxanes such as those sold under the name PS560-K6 by UCT (United Chemical Technologies Inc.) or under the name MCR-M17 by Gelest Inc.

The macromonomers are preferably present in the polymers of the invention in a proportion of from 2% to 16% by weight, preferably from 4% to 15% by weight and better still from 6% to 12% by weight, and even better still from 8% to 10% by weight relative to the total weight of the said polymer.

Polymers that are particularly advantageous according to the invention are those obtained by polymerization of:

methyl acrylate and of a monomethacryloyloxypropyl-polydimethylsiloxane macromonomer, especially of Mw from 800 to 5000, especially in decamethylcyclopentasiloxane or phenyl trimethicone; or

methyl acrylate, acrylic acid and of a monomethacryloyloxy-propylpolydimethylsiloxane macromonomer, especially of Mw from 800 to 5000, especially in decamethylcyclopentasiloxane or phenyl trimethicone.

The weight-average molecular mass (Mw) of the polymer is preferably between 10,000 and 300,000, especially between 20,000 and 200,000 and better still between 25,000 and 150,000

By virtue of the abovementioned characteristics, in a given silicone medium, polymers of the invention have the capacity of folding over on themselves, thus forming solid particles of substantially spherical shape, the periphery of these particles having the deployed side chains, which ensure the stability of these particles. Such particles resulting from the characteristics of the polymers of the invention have the particular feature of not aggregating in the said medium and thus of being self-stabilized and of forming a particularly stable polymer particle dispersion.

In particular, polymers of the invention are capable of forming nanometer-sized particles, with a mean size ranging from 10 to 400 nm and preferably from 20 to 200 nm.

As a result of this very small size, the particles forming part of the constitution of the dispersion are particularly stable and therefore have little susceptibility to form aggregates.

The dispersion of the invention is thus a dispersion that is stable in the medium under consideration and does not form sediments when it is placed at room temperature (approximately 25° C.) for an extended period (for example 24 hours).

Preferably, the particle dispersion has a solids content (or dry extract) of from 40% to 70% by weight of solids and especially from 45% to 65% by weight.

The said polymer or the said polymer particle dispersion may be prepared via a process comprising a step consisting in performing a free-radical copolymerization, in a medium corresponding to the definition given above, of one or more acrylic monomers as defined above with one or more silicone macromonomers as defined above.

The copolymerization may be performed conventionally in the presence of a polymerization initiator. The polymerization initiators may be free-radical initiators. In general, such a polymerization initiator may be chosen from organic peroxide compounds such as dilauroyl peroxide, dibenzoyl peroxide or tert-butyl peroxy-2-ethylhexanoate; and diazo compounds such as azobisisobutyronitrile or azobisdimethylvaleronitrile.

The reaction may also be initiated using photoinitiators or with radiation such as UV or neutrons, or with plasma.

In general, to perform this process, at least a portion of the silicone medium, a portion of the acrylic and/or additional monomers, which will constitute the insoluble skeleton after polymerization, all of the macromonomer (which will constitute the side chains of the polymer), and a portion of the polymerization initiator are introduced into a reactor whose size is suitable for the amount of polymer to be prepared. At this stage of introduction, the reaction medium forms a relatively homogeneous medium.

The reaction medium is then stirred and heated up to a temperature to obtain polymerization of the monomers and macromonomers. After a certain time, the initially homogeneous and clear medium leads to a dispersion of milky appearance. A mixture consisting of the remaining portion of monomer and of polymerization initiator is then added. After an adequate time during which the mixture is heated with stirring, the medium stabilizes in the form of a milky dispersion, the dispersion comprising polymer particles stabilized in the medium in which they have been created, the said stabilization being due to the presence of side chains that are soluble in the said medium.

It is also possible to prepare the polymer particle dispersion in a polymerization medium different from the silicone medium, wherein the said different medium is further replaced, after polymerization, by the non-aqueous silicone medium according to the invention.

The polymer particle dispersion according to the invention may be used in any type of composition and especially in a cosmetic or pharmaceutical composition comprising a cosmetically or pharmaceutically acceptable medium, such as a care, cleansing or makeup composition for the skin or keratin materials, a haircare composition or an anti-sun composition.

The dispersion may be present in a proportion of from 3% to 95% by weight in the composition, especially 4-90% by weight or even 20-70% by weight.

Preferably, the composition comprises from 0.5% to 25% by weight, especially from 1% to 20% by weight, more specially from 4% to 17% by weight and most preferably from 5% to 15% by weight of dry matter of polymer according to the invention, relative to the total weight of the composition.

Depending on the desired application, the composition may contain adjuvants commonly incorporated into cosmetic or pharmaceutical compositions.

Among these adjuvants that may be mentioned are fatty substances, and especially waxes, oils, gums and/or pasty fatty substances, which are hydrocarbon-based and/or silicone-based, and pulverulent compounds such as pigments, fillers and/or nacres.

Among the waxes that may be present in the composition according to the invention, mention may be made, alone or as a mixture, of hydrocarbon-based waxes such as beeswax, carnauba wax, candelilla wax, ouricury wax, Japan wax, cork fibre wax or sugar cane wax; paraffin wax; lignite wax; microcrystalline waxes; lanolin wax; montan wax; ozokerites; polyethylene waxes; the waxes obtained by Fischer-Tropsch synthesis; and hydrogenated oils, fatty esters and glycerides that are solid at 25° C. Silicone waxes may also be used, among which mention may be made of alkyl and alkoxy polymethylsiloxanes, and/or polymethylsiloxane esters.

Among the oils that may be present in the composition according to the invention, mention may be made, alone or as a mixture, of hydrocarbon-based oils such as liquid paraffin or liquid petroleum jelly; perhydrosqualene; arara oil; sweet almond oil; beauty-leaf oil; palm oil; castor oil; avocado oil; jojoba oil; olive oil or cereal germ oil; lanolic acid; oleic aci; lauric acid or stearic acid esters; and alcohols such as oleyl alcohol, linoleyl alcohol, linolenyl alcohol, isostearyl alcohol or octyldodecanol. Mention may also be made of silicone oils such as optionally phenylated PDMSs, such as phenyl trimethicones. Mention may also be made of volatile oils, such as cyclotetradimethylsiloxane, cyclopentadimethylsiloxane, cyclohexadimethylsiloxane, methylhexyldimethylsiloxane, hexamethyldisiloxane and isoparaffins.

The pigments may be white or coloured, and mineral and/or organic. Among the mineral pigments that may be mentioned are titanium dioxide, zirconium dioxide or cerium dioxide, and also zinc oxide, iron oxide or chromium oxide, and ferric blue. Among the organic pigments that may be mentioned are carbon black and barium, strontium, calcium or aluminium lakes.

The nacres may be chosen from mica coated with titanium oxide, with iron oxide, with natural pigment or with bismuth oxychloride, and also coloured titanium mica.

The fillers may be lamellar or non-lamellar, and mineral or synthetic. Mention may be made of talc, mica, silica, kaolin, nylon powder, polyethylene powder, Teflon, starch, titanium mica, natural nacre, boron nitride, hollow microspheres such as Expancel (Nobel Industrie), Polytrap (Dow Corning) and silicone resin microbeads (for example Tospearls from Toshiba).

The composition may also comprise any additive usually used in cosmetics, such as antioxidants; fragrances; essential oils; preserving agents; cosmetic active agents; moisturizers; vitamins; essential fatty acids; sphingolipids, sunscreens, surfactants, and liposoluble polymers, for instance polyalkylenes, especially polybutene, polyacrylates and silicone polymers that are compatible with fatty substances. Needless to say, a person skilled in the art will take care to select this or these optional additional compound(s), and/or the amount thereof, such that the advantageous properties of the composition according to the invention are not, or are not substantially, adversely affected by the envisaged addition.

The compositions according to the invention may be in any form that is acceptable and usual for a cosmetic, hygiene or pharmaceutical composition, and especially in the form of an oil-in-water or water-in-oil emulsion, a lotion, a mousse or a spray.

Among the applications preferably targeted by the present invention, mention may be made more particularly of:

the field of haircare products (washing, care or beauty of the hair), the compositions according to the invention being in particular in the form of aerosols, mousses, shampoos, conditioners, styling or treating lotions or gels, and shaping, hairsetting or fixing lacquers or lotions;

the field of makeup products, in particular for making up the eyelashes, the compositions being in the form of mascara or eyeliner, lipstick, lip gloss, foundation, makeup rouge or eyeshadow; and

the field of care products for body skin and facial skin, especially anti-sun products or self-tanning products.

A subject of the present invention is also a cosmetic treatment process for caring for, cleansing and/or making up keratin materials such as the skin, the scalp, the eyelashes, the eyebrows, the lips or the nails, comprising applying a composition as defined above to the said keratin materials.

The invention will now be described in greater detail in the light of the examples that follow, which are given as non-limiting illustrations.

The present examples illustrate the preparation of polymers in accordance with the invention, capable of forming a dispersion of particles in a given medium.

In these examples, the weight-average molar mass (Mw) and number-average molar mass (Mn) of the polymer, the glass transition temperature of the polymer, the solids content (or dry extract) of the dispersion and size of the polymer particles are determined, after preparation of the said dispersion.

The weight-average (Mw) and number-average (Mn) molar masses are determined by gel-permeation liquid chromatography (THF solvent, calibration curve established with linear polystyrene standards, refractometric detector).

The measurement of the glass transition temperature (Tg) is performed according to standard ASTM D3418-97, by differential thermal analysis (DSC “Differential Scanning Calorimetry”) on a calorimeter, over a temperature range of between −100° C. and +150° C., at a heating rate of 10° C./minute in 150 μl aluminium crucibles.

The crucibles are prepared in the following manner: 100 μl of the dispersion obtained are introduced into a 150 μl aluminium crucible and the solvent is allowed to evaporate over 24 hours at room temperature and at 50% RH. The operation is repeated and the crucible is then introduced into a Mettler DSC30 calorimeter.

The solids content (or dry extract), i.e. the amount of non-volatile matter, may be measured in various ways: mention may be made, for example, of the methods by oven-drying or the methods by drying by exposure to infrared radiation.

The solids content is preferably measured by heating the sample with infrared rays with a wavelength of from 2 μm to 3.5 μm. The substances contained in the composition that have a high vapour pressure evaporate under the effect of this radiation. Measuring the weight loss of the sample makes it possible to determine the dry extract of the composition. These measurements are performed using an LP16 commercial infrared desiccator from Mettler. This technique is fully described in the documentation for the machine supplied by Mettler.

The measuring protocol is as follows: about 1 g of the composition is spread onto a metal cup. After introducing this cup in the desiccator, it is subjected to a nominal temperature of 120° C. for 1 hour. The wet mass of the sample, corresponding to the initial mass, and the dry mass of the sample, corresponding to the mass after exposure to the radiation, are measured using a precision balance.

The solids content is calculated in the following manner:

dry extract=100×(dry mass/wet mass).

The particle sizes may be measured by various techniques: mention may be made in particular of light-scattering techniques (dynamic and static), Coulter counter methods, sedimentation rate measurements (related to the size via Stokes' law) and microscopy. These techniques make it possible to measure a particle diameter and, for some of them, a particle size distribution.

The sizes and size distributions of the particles in the compositions according to the invention are preferably measured by static light scattering using a commercial granulometer such as the MasterSizer 2000 from Malvern. The data are processed on the basis of the Mie scattering theory. This theory, which is exact for isotropic particles, makes it possible to determine an “effective” particle diameter in the case of non-spherical particles. This theory is described especially in the publication by Van de Hulst, H. C., “Light Scattering by Small Particles,” Chapters 9 and 10, Wiley, New York, 1957.

The composition is characterized by its mean “effective” diameter by volume D[4.3], defined in the following manner:

$$D [ 4.3 ] = \frac{{\sum }_{i} {V}_{i} \cdot {d}_{i}}{{\sum }_{i} {V}_{i}}$$

in which Vᵢ represents the volume of the particles with an effective diameter dᵢ. This parameter is described especially in the technical documentation of the granulometer.

The measurements are performed at 25° C. on a dilute particle dispersion, obtained from the composition in the following manner: 1) dilution by a factor of 100 with water, 2) homogenization of the solution, 3) standing of the solution for 18 hours, 4) recovery of the whitish uniform supernatant.

The “effective” diameter is obtained by taking a refractive index of 1.33 for water and a mean refractive index of 1.42 for the particles.

### EXAMPLE 1

This example illustrates the preparation of a polymer forming a particle dispersion in a silicone solvent, the said polymer being obtained by polymerization of methyl acrylate in the presence of monomethacryloxypropyldimethoxysilane macromonomer (PS560-K6).

150 g of decamethylcyclopentasiloxane, 12.75 g of methyl acrylate, 2.25 g of monomethacryloxypropyl-polydimethylsiloxane (PS560-K6 of Mw=1,000) and 0.8 g of tert-butyl peroxy-2-ethylhexanoate (Trigonox 21S) are placed in a 500 ml reactor.

The reaction mixture is stirred and heated to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 35 g of methyl acrylate and 0.5 g of Trigonox 21S. Heating is then continued for 4 hours at 90° C., after which the heptane is distilled from the reaction medium. After this heating operation, a stable dispersion of polymer particles in decamethylcyclopentasiloxane (D5) is obtained.

The characteristics of the polymer and of the particles formed by the said polymer are as follows:

weight-average molecular mass Mw: 186,800

number-average molecular mass Mn: 36,830

polydispersity index (Mw/Mn)=5.07

glass transition: 7° C. by Mettler DSC

dry extract: 25% in D5, performed by thermal balance

particle size: 160 nm with polydispersity of 0.05, performed on a Malvern Autosizer Lo-C at 25° C.

The macromonomer represents 4.5% by weight of the polymer.

The stability of the dispersion obtained is demonstrated by performing the following stability protocol: 8 ml of the prepared dispersion are placed in a haemolysis tube and are centrifuged at 4,000 rpm for 15 minutes using a Jouan C100-S5 centrifuge. After 15 minutes, it is noted that there is no phase separation, which demonstrates that the dispersion is stable.

### EXAMPLE 2

This example illustrates the preparation of a polymer forming a particle dispersion in a silicone solvent, the said polymer being obtained by polymerization of methyl acrylate in the presence of a monomethacryloxypropylpolydimethylsiloxane (MCR-M17) macromonomer.

200 g of heptane, 200 g of decamethylcyclopentasiloxane, 30 g of methyl acrylate, 10 g of monomethacryloxypropylpolydimethylsiloxane macromonomer (MCR-M17 of Mw=5,000) and 3.2 g of tert-butyl peroxy-2-ethylhexanoate (Trigonox 21S) are placed in a 1 liter reactor.

The reaction mixture is stirred and heated to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 160 g of methyl acrylate and 2 g of Trigonox 21S. Heating is then continued for 4 hours at 90° C., after which the heptane is distilled from the reaction medium.

After this distillation operation, a stable dispersion of polymer particles thus prepared in the decamethylcyclopentasiloxane (D5) is obtained.

The characteristics of the polymer and of the particles formed by the said polymer are as follows:

weight-average molecular mass Mw: 102,347

number-average molecular mass Mn: 28,283

polydispersity index (Mw/Mn)=3.62

dry extract: 51.4% in D5, performed by thermal balance

glass transition: 12° C. by Mettler DSC

particle size: 160 nm with polydispersity of 0.04, performed on a Malvern Autosizer Lo-C at 25° C.

The macromonomer represents 5% by weight of the polymer.

After performing the stability protocol in accordance with Example 1, it is found that the dispersion is stable.

### EXAMPLE 3

This example illustrates the preparation of a polymer forming a particle dispersion in a silicone solvent, the said polymer being obtained by polymerization of methyl acrylate and of monomethacryloxypropylpolydimethylsiloxane macromonomer (MCR-M17).

200 g of heptane, 200 g of decamethylcyclopentasiloxane, 30 g of methyl acrylate, 16 g of monomethacryloxypropylpolydimethylsiloxane macromonomer (MCR-M17 of Mw=5,000) and 3.2 g of tert-butyl peroxy-2-ethylhexanoate (Trigonox 21S) are placed in a 1 liter reactor.

The reaction mixture is stirred and heated to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 154 g of methyl acrylate and 2 g of Trigonox 21S. Heating is then continued for 4 hours at 90° C., after which the heptane is distilled from the reaction medium.

After this distillation operation, a stable dispersion of polymer particles thus prepared in the decamethylcyclopentasiloxane is obtained.

The characteristics of the polymer and of the particles formed by the said polymer are as follows:

weight-average molecular mass Mw: 118,986

number-average molecular mass Mn: 29,914

polydispersity index (Mw/Mn)=3.98

dry extract: 49.6% in D5, performed by thermal balance

glass transition: 12° C. by Mettler DSC

particle size: 170 nm with polydispersity of 0.04, performed on a Malvern Autosizer Lo-C at 25° C.

The macromonomer represents 8% by weight of the polymer.

After performing the stability protocol in accordance with Example 1, it is found that the dispersion obtained is stable.

### EXAMPLE 4

This example illustrates the preparation of a polymer forming a particle dispersion in a silicone solvent, the said polymer being obtained by polymerization of methyl acrylate and of monomethacryloxypropylpolydimethylsiloxane macromonomer (MCR-M17).

200 g of heptane, 200 g of phenyl trimethicone, 30 g of methyl acrylate, 10 g of monomethacryloxypropylpolydimethylsiloxane macromonomer (MCR-M17) of Mw=5,000) and 3.2 g of tert-butyl peroxy-2-ethylhexanoate (Trigonox 21S) are placed in a 1 liter reactor.

The reaction mixture is stirred and heated to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 160 g of methyl acrylate and 2 g of Trigonox 21S. Heating is then continued for 4 hours at 90° C., after which the heptane is distilled from the reaction medium.

After this distillation operation, a stable dispersion of polymer particles thus prepared in phenyl trimethicone is obtained.

The characteristics of the polymer and of the particles formed by the said polymer are as follows:

weight-average molecular mass Mw: 95,630

number-average molecular mass Mn: 25,690

polydispersity index (Mw/Mn)=3.72

theoretical dry extract: 50% in phenyl trimethicone

glass transition: 12° C. by Mettler DSC

particle size: 150 nm with polydispersity of 0.04, performed on a Malvern Autosizer Lo-C at 25° C.

The macromonomer represents 5% by weight of the polymer.

After performing the stability protocol in accordance with Example 1, it is found that the dispersion obtained is stable.

### EXAMPLE 5

This example illustrates the preparation of a polymer forming a particle dispersion in a silicone solvent, the said polymer being obtained by polymerization of methyl acrylate, of acrylic acid and of monomethacryloxypropyldimethylsiloxane macromonomer (MCR-M17).

200 g of heptane, 200 g of D5, 26 g of methyl acrylate, 14 g of monomethacryloxypropylpolydimethylsiloxane macromonomer (MCR-M17 of Mw=5,000) and 3.2 g of tert-butyl peroxy-2-ethylhexanoate (Trigonox 21S) are placed in a 1 liter reactor.

The reaction mixture is stirred and heated to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 120 g of methyl acrylate, 40 g of acrylic acid and 2 g of Trigonox 21S. Heating is then continued for 4 hours at 90° C., after which the heptane is distilled from the reaction medium.

After this distillation operation, a stable dispersion of polymer particles is thus prepared in D5 is obtained.

The characteristics of the polymer and of the particles formed by the said polymer are as follows:

theoretical dry extract: 50% in D5, performed by thermal balance

glass transition: 12° C. by Mettler DSC

particle size: 170 nm with polydispersity of 0.04, performed on a Malvern Autosizer Lo-C at 25° C.

The macromonomer represents 7% by weight of the polymer.

After performing the stability protocol in accordance with Example 1, it is found that the dispersion obtained is stable.

### EXAMPLE 6

### Mascara Composition

A mascara having the composition below was prepared:

| | | |
|---|---|---|
| Beeswax | 8 | g |
| Paraffin wax | 3 | g |
| Carnauba wax | 6 | g |
| Hectorite modified with | 5.3 | g |
| distearyldimethylbenzylammonium chloride | | |
| (Bentone ® 38 V from Elementis) | | |
| Propylene carbonate | 1.7 | g |
| Filler | 1 | g |
| Pigments | 5 | g |
| Polymer dispersion of Example 4 | 12 | g AM* |
| Isododecane | qs 100 | g |

*AM: active material

After application to the eyelashes, the mascara is considered as very satisfactory.

### EXAMPLE 7

### Stick of Lipstick

The lipstick composition below is prepared:

| | |
|---|---|
| Polyethylene wax | 15% |
| Polymer dispersion of Example 3 | 10% AM |
| Hydrogenated polyisobutene (Parleam from | 26% |
| Nippon Oil Fats) | |
| Pigments | 8.6% |
| Isododecane | qs 100% |

After application to the lips, the composition obtained shows good cosmetic properties.

### EXAMPLE 8

### W/O Foundation

A foundation composition comprising the compounds below is prepared:

| | | | |
|---|---|---|---|
| Phase A | Cetyldimethicone copolyol | 3 | g |
| | (Abil EM90 from the company | | |
| | Goldschmidt) | | |
| | Isostearyl diglyceryl succinate | 0.6 | g |
| | (Imwitor 780K from the company | | |
| | Condea) | | |
| | Isododecane | 18.5 | g |
| | Mixture of pigments | 10 | g |
| | (hydrophobic iron oxides and | | |
| | titanium oxides) | | |
| | Polymer dispersion of Example 3 | 8.7 | g AM |
| | Polyamide powder (Nylon-12 from | 8 | g |
| | Dupont de Nemours) | | |
| | Fragrance | qs | |
| Phase B | Water | qs 100 | g |
| | Magnesium sulphate | 0.7 | g |
| | Preserving agent | qs | |
| | (methylparaben) | | |
| Phase C | Water | 2 | g |
| | Preserving agent | qs | |
| | (diazolinylurea) | | |

The composition obtained shows good cosmetic properties.

### EXAMPLE 9

### Compacted Powder

A compacted powder having the composition below is prepared:

Composition A:

| | | |
|---|---|---|
| Talc | 30 | g |
| Bismuth oxychloride | 10 | g |
| Zinc stearate | 4 | g |
| Nylon powder | 20 | g |
| Dispersion of Example 1 | 5 | g |

Composition B:

| | | |
|---|---|---|
| Iron oxides | 2 | g |
| Liquid petroleum jelly | 6 | g |

The powder is obtained in the following manner: composition A is ground in a Kenwood mill for about 5 minutes with gentle stirring, composition B is added and the mixture is ground for about 2 minutes at the same speed, and then for 3 minutes at a higher speed. The preparation is then screened through a 0.16 mm screen, and this mixture is then compacted in dishes.

A compacted powder with good cosmetic properties is obtained.

The composition obtained is easy and pleasant to apply. It is found that the film does not migrate into the fine lines of the skin, even after having been worn for several hours.

### EXAMPLE 10

### Facial Gel

The composition below is prepared:

| | | |
|---|---|---|
| Isopropyl palmitate | 10 | g |
| Petroleum jelly (wax) | 5 | g |
| Modified hectorite (clay) | 0.15 | g |
| Ozokerite (wax) | 5 | g |
| Oxyethylenated sorbitan septaoleate (40 OE) | 5 | g |
| Dispersion of Example 4 (25% solids) | 75 | g |

A gel with good cosmetic properties is obtained.

### EXAMPLE 11

### Care Oil

The composition below is prepared:

| | |
|---|---|
| Dispersion of Example 2 (25% solids) | 70 g |
| Jojoba oil | 15 g |
| Soybean oil | 15 g |

A care oil that may be applied to the body or the face is obtained.

We claim:

1. A dispersion of particles in a non-aqueous, silicone medium wherein said particles comprise at least one acrylic polymer comprising:
(A) a skeleton that is insoluble in said medium; and
(B) a portion of said polymer that is soluble in said medium comprising side chains covalently bonded to said skeleton, wherein said polymer is obtained by polymerization of a polymerizable mixture, comprising:
(i) a first C₁-C₃ alkyl (meth)acrylate monomer, alone or as a mixture of C₁-C₃ alkyl (meth)acrylate monomers, optionally in the presence of one or more additional monomers selected from the group consisting of acrylic acid, methacrylic acid and alkyl (meth)acrylates of formula (I), 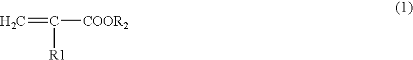
and the salts thereof, wherein:
R₁ is a hydrogen atom or a methyl group; and
R₂ is:
(a) a linear or branched alkyl group containing from 1 to 6 carbon atoms, said group containing one or more substituents selected from the group consisting of one or two oxygen atoms, —OH, F, Cl, Br, I, and —NR′R″, wherein R′ and R″, which may be identical or different, are linear or branched C₁-C₃ alkylgroups; or
(b) a cyclic alkyl group containing from 3 to 6 carbon atoms, said group optionally containing one or more oxygen atoms, and optionally containing one or more substituents selected from the group consisting of OH, F, Cl, Br, and I; and
(ii) at least one silicone macromonomer comprising an end group that reacts during said polymerization said side chains, said macromonomer having a weight-average molecular mass of at least 200 and representing 0.05% to 20% by weight of the polymer.

2. The dispersion of claim 1, wherein said non-aqueous silicone medium comprises at least 50% by weight of at least one non-aqueous silicone liquid compound having a global solubility parameter according to the Hansen solubility space of less than or equal to 17 (MPa)^{1/2}.

3. The dispersion of claim 1, wherein said first monomer, or mixture of first monomers, is present in an amount of 50-100% by weight of the mixture of first monomer(s) and optional additional monomer(s).

4. The dispersion of claim 1, wherein said first monomer, or mixture of first monomers, is selected from the group consisting of methyl acrylate; methyl methacrylate; ethyl acrylate; ethyl methacrylate; n-propyl acrylate; n-propyl methacrylate; isopropyl acrylate; and isopropyl methacrylate.

5. The dispersion of claim 1, wherein said one or more additional monomers, or mixture of additional monomers, is selected from the group consisting of methoxyethyl (meth)acrylate; ethoxyethyl (meth)acrylate; trifluoroethyl methacrylate; dimethylaminoethyl methacrylate; diethylaminoethyl methacrylate; 2-hydroxypropyl methacrylate; 2-hydroxyethyl methacrylate; 2-hydroxypropyl acrylate; 2-hydroxyethyl acrylate; and the salts thereof.

6. The dispersion of claim 1, wherein said silicone macromonomer comprises an end group selected from the group consisting of a vinyl group and a (meth)acryloyloxy group.

7. The dispersion of claim 1, wherein said silicone macromonomer has a weight-average molecular mass (Mw) from 200 to 100,000.

8. The dispersion of claim 14, wherein said weight-average molecular mass (Mw) is from 300 to 50,000.

9. The dispersion of claim 1, wherein said silicone macromonomer comprises a polydimethylsiloxane containing a monoacryloyloxy or monomethacryloyloxy end group.

10. The dispersion of claim 1, wherein said silicone macromonomer is a compound of formula (II) 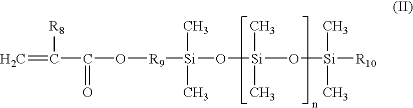
wherein:
R₈ is a hydrogen atom or a methyl group;
R₉ is a divalent linear or branched hydrocarbon group containing from 1 to 10 carbon atoms, said group optionally containing one or two oxygen atoms;
R₁₀ is a linear or branched alkyl group containing from 1 to 10 carbon atoms; and
n is an integer from 1 to 300.

11. The dispersion of claim 10, wherein R₈ is a methyl group.

12. The dispersion of claim 10, wherein R₉ is selected from the group consisting of ethylen, propylen, and butylen.

13. The dispersion of claim 10, wherein R₁₀ is selected from the group consisting of methyl, ethyl, propyl, butyl, and pentyl.

14. The dispersion of claim 1, wherein said silicone macromonomer is present in the polymer in a proportion of from 2-16% by weight.

15. The dispersion of claim 14, wherein said proportion is from 4-15% by weight.

16. The dispersion of claim 1, wherein said acrylic polymer has a weight-average molecular mass (Mw) of between 10,000 and 300,000.

17. The dispersion of claim 16, wherein said weight-average molecular mass (Mw) is between 20,000 and 200,000.

18. The dispersion of claim 1, wherein said polymer particles have a mean size ranging from 10 to 400 nm.

19. The dispersion of claim 1, wherein said dispersion has a solids content (or dry extract) of from 40-70% by weight of solids.

20. A cosmetic or pharmaceutical composition, comprising a dispersion according to claim 1 and a cosmetically or pharmaceutically acceptable medium.

21. The composition of claim 20, wherein said dispersion is present in an amount of from 3-95% by weight of said composition.

22. The composition of claim 20, wherein said cosmetically or pharmaceutically acceptable medium comprises one or more substances selected from the group consisting of waxes; oils; gums; pasty fatty substances; pigments; fillers; nacres; antioxidants; fragrances; essential oils; preserving agents; cosmetic active agents; moisturizers; vitamins; essential fatty acids; sphingolipids; sunscreens; surfactants; and liposoluble polymers compatible with fatty substances.

23. The composition of claim 20, which is in the form of a care, cleansing or makeup composition for the skin or keratin materials, a haircare composition, or an anti-sun composition.

24. A cosmetic treatment process for caring for, cleansing and/or making up keratin materials such as the skin, the scalp, the eyelashes, the eyebrows, the lips and the nails, comprising applying the composition of claim 20 to said keratin materials.
